(19) United States
(12) Patent Application Publication
(10) Pub. No.: US 2008/0152609 A1
(43) Pub. Date: Jun. 26, 2008

(54) Suppression of sulfer fumes by higher boiling point oils

(76) Inventor: John Jacob Wille, Chesterfield, NJ (US)

Correspondence Address: MILDE & HOFFBERG, LLP, 10 BANK STREET, SUITE 460, WHITE PLAINS, NY, 10606, US

(21) Appl. No.: 11/998,698

(22) Filed: Nov. 30, 2007

Related U.S. Application Data
(60) Provisional application No. 60/873,717, filed on Dec. 8, 2006.

(51) Int. Cl. A61K 8/65; A61Q 5/04

(52) U.S. Cl. 424/70.5

(57) ABSTRACT

A method is disclosed for suppressing low vapor pressure sulfurous fumes generated by sulfur-containing organic compounds, such as is found in personal hair care products. The method includes the step of applying a liquid with a higher vapor pressure temperature than the sulfur-containing compounds to materials contaminated with the sulfur-containing organic compounds.

### RELATED APPLICATIONS

This application claims priority from provisional Application 60/873,717 filed Dec. 8, 2006, entitled “Suppression of Sulfur fumes by Higher boiling Point Oils.”

### FIELD OF THE INVENTION

The field of the invention is in personal care, environmental biotechnology, health care, and more specifically in consumer household products that are marketed for elimination of noxious fumes.

### BACKGROUND OF THE INVENTION

Sulfur fumes represent one of the worst environmental problems to out-door air quality and where household and personal care products generate sulfur fumes. In particular, the use of thioglycolic acid during the hair-conditioning step of the permanent wave process is often accompanied by the generation of malodorous sulfur fumes. Suppression of these fumes would be a major benefit to hair salon operators, their workers and most desirable for improved customer satisfaction.

Another object of the this invention concerns the elimination of sulfur fumes from home heating oil, diesel fuels and gasoline spills that pose a serious challenge as environmental pollutants when they enter the environment. Petroleum oils contain significant amount of sulfur compounds that contribute to the toxic odor of oil spills. Oil spills that occur in the open waters are primarily dealt with by immediate and proper notification of agencies that require the responsible parties to take action as required by statutes at the local and state levels. For major oil spills the polluted areas must be cleaned up within 48 hours by government approved immediate response teams. These clean up efforts are monitored by the Environmental Pollution Agency (EPA) and must meet standard guidelines for attaining minimal residual oil levels.

Oil spills in open waters from tanker operations are regulated by international convention whether they occur as a accidental spill or by oil discharge from engine maintenance and machinery used in ordinary ship operations. The oil spill standard is set at less than 10 ppm seen in open waters as a slight oil sheen. All of these sources of oil spills can indirectly result in minor petrolatum hydrocarbon oil contamination of work clothes and skin by ship personnel. It is this latter source of fumes that the present invention is directed.

U.S. Pat. No. 6,267,888 discloses a method for removal of free-floating oil from water by biodispersion and bioutilization. The method employs a living mixture of bacterial species that have the ability to utilize hydrocarbons as the only source of carbon in an oleophilic liquid vehicle that provides oil soluble source of nitrogen and phosphorus for the bacteria. In principle, petroleum hydrocarbon fumes are remediated by the bacteria consuming any residual oil.

U.S. Pat. No. 4,237,780 describes a method for disposal of hydrocarbon fumes through the use of a prefilter and filter made from wood chips and carbonized wood chips, respectively. The primary or intended application of this invention is for use in paint spray booths to dispose of noxious organic solvents that contribute to air pollution.

The use of fans to vent the fumes to the outside air is a common practice for hydrocarbon oil fumes in oil-polluted basements. Unfortunately, this practice rarely is completely effective as oil residues can persist in porous concrete and wood that slowly releases the fumes over long period of time.

U.S. Pat. No. 6,398,960 discloses a method for remediating aquifers and groundwater contaminated by toxic halogenated organic compounds, certain inorganic compounds, and oxidized heavy metals and radionuclides. They teach the use of innocuous oil preferably edible food grade oil such as soybean oil, formulated in a microemulsion. We include their literature reference list here by way of its teaching that the use of vegetable oil is based on its supposed acceleration of reductive dehalogenation via a chemical reaction mechanism.

The aforementioned methods for treating sulfur-containing hydrocarbon fumes are designed primarily for remediation of either free-floating oil in water or hydrocarbon residues that have entered into aquifers and ground water. They do not address oil contamination of human body skin. In particular, the patent art does not pertain to methods for decontamination of oil and gasoline contamination of personal effects such as clothes, household or office furniture and carpeting. There also remains one of the problems of concern that is the focus in the present invention, i.e., hydrocarbon fumes associated with minor home heating oil spills in home basements, and commercial or private machine shops. Still another source of harmful petroleum fumes occurs during routine filling of auto gasoline tanks and diesel oil spilled on hands and clothing during the filling and maintenance of autos, road vehicle, recreational off-road vehicles, pleasure boats, and ordinary household equipment such as lawn tractors and other garden equipment.

In the case of gasoline spills on hands or clothing the gasoline fumes persist even after meticulous cleansing of the affected areas with ordinary soap and water. This situation is all too frequent and unfortunately there is no available product on the market to meet this unmet consumer need. Likewise in the case where homeowners are confronted with noxious sulfur-containing heating oil fumes due to oil furnace maintenance work, repairs or accidental spills, the fumes can permeate the entire house posing a serious health problem for the inhabitants. Typically, heating oil spills have been cleaned up with old rags, absorbent pads and wipes that may clean up the overt spilled oil but fail to eliminate oil fumes that remain in the concrete or sand surface. These unseen oil spills still emit noxious fumes and often require vacating the domicile until more extensive excavations and removal of contaminated materials. This can be costly and time-consuming form the homeowner and may pose future problems to the homeowner in meeting petroleum oil spill standards preparatory to resale of the property. Still another problem is the inadvertent contamination of furniture and household carpeting by petroleum hydrocarbon oils. Ordinary detergent-based cleansers fail to completely eliminate fumes from these hydrocarbon oil spills as long as they remain embedded in the soiled material.

Many household products are available to rid malodors arising from microbial action on organic wastes associated with bathrooms and kitchens. They generally provide a temporary solution by masking malodors through devices that release of a pleasing scent or fragrance. The most common method listed by a recent survey in Happi (Household and Personal Products Industry Magazine, September, 2004) is the burning of scented candles, followed closely by scented air fresheners. Again, these methods do not suppress malodors or prevent their escape into the air. The present invention discloses a novel and unobvious solution to minor sulfur containing petroleum hydrocarbon fumes that not merely masks or perfumes the fumes but permanently suppresses their vaporization from the oil-affected sites.

Currently, solutions to the reduction or elimination of these sulfur-borne fumes are limited to scrubbers and precipitators for coal burning plants, which are inadequate and inappropriate to reducing the fumes generated by the use of personal care products, or other small scale fume generation of sulfur. For example, in the use of a permanent wave hair product, both the application of the product to the hair, and heat required to activate the product, exacerbate the formation of sulfur fumes from liquid thioglycolic acid containing product formulation. In U.S. Pat. No. 6,302,119 disclose the use of disulfides before, during and after application of sulfur-based reducing agents as a method to reduce odors associated with permanent hair waving. Unfortunately a chemical means of reacting the sulfur odors by reducing agents may interfere with the waving process. U.S. Pat. No. 6,403,642 discloses the use of compositions for absorbing sulfur-containing compounds and for elimination or reducing odors associated with ingestion of foods or medicines that cause sulfur odors. Such compositions contain a metal complex of a substrate and a ligand that may comprise an amino acid containing sulfur or a carboxylic acid such as cystine. In another disclosure Shacknai et al., claim a method for reducing the production of malodor in sulfur containing dermatological compositions by adjusting the pH of the composition to be between 6.5 and 8.1.

This method, like those discussed above, depends on altering the chemical reactivity of sulfur odor emitting agents. Therefore, there is a commercial need for a non-reducing agent that is fast, inexpensive and a complete method to reduce sulfur fumes without altering the chemical reactivity or masking the smell by the use of masking agent such as fragrances and perfumes.

### SUMMARY OF THE INVENTION

The principal objective of the present invention is to provide a method for rapidly and permanently suppressing low vapor pressure (less than 1 atmosphere) sulfurous fumes.

The method involves the application of a vegetable fatty acid with a higher boiling point than the sulfur odor generating chemicals that are characterized by a lower vapor pressure.

More particularly, the present invention concerns methods and formulations that effectively suppress volatile sulfur fumes. The concept arose from observations made by the inventor that the application of oleophilic liquid vehicle to sulfur-containing organic solutions quickly damped the fumes and replaced it with a vegetable-like odor. These observations were repeated a number of time and led to a quest to determine if a simpler system of vegetable oils had the property of suppressing petroleum hydrocarbon fumes. This search revealed that fatty acid vegetable oils such as oleic acid were very effective in eliminating diesel oil fumes applied to various substrates. The scientific, technical and rational basis for this property was further explored and a general principle emerged from these studies. We formed the hypothesis that sulfur fume suppression occurs when oil with a relatively higher vapor pressure temperature (non-fume producer) is applied to the surface of a sulfur-containing organic liquid with a lower vapor pressure temperature (volatile fume producer). The Chemical Rubber Publishing Company's Handbook of Physics and Chemistry, 35th Edition, 1953, defines vapor pressure temperature as the values for the temperature in degrees centigrade at which the vapor of the compound has the pressure indicated in the corresponding table of compounds. For one (1) atmosphere that is a pressure of 760 mm of Mercury, and for organic compounds with pressures less than one atmosphere and carbon-atom chain length below C29, the vapor pressure temperature data are found in the Handbook of Chemistry and Physics 35^{th} Edition, pp. 2177-2225.

A rule of prediction was developed. In order to find a given fatty acid oil that will predictably suppress organic sulfur fumes reference is made to the vapor pressure temperature of their primary constituents. These values are founds in CRC Handbook of Chemistry and Physics, 35th Edition, pp 2177-2225). Once identified, it only remains to make reference to the vapor pressure temperature of fatty acid vegetable oils that have a notably higher vapor pressure temperature.

### Chemistry of Vapor Suppression

Research has confirmed our hypothesis that suppression of sulfur fumes generated from sulfur containing organic compounds with a given volatile vapor occurs when miscible oils, with a vapor pressure temperature greater than the given volatile organic sulfur vapor pressure temperatures, is applied to the sulfur containing organic compounds. For example, 2-mercaptoacetic acid has a vapor pressure temperature of 104° C. at 100 mm of Mercury pressure, which is well below the vapor pressure temperature of oleic acid at 100 mm Mercury.

This method explains in simple mathematical terms the success of applying oleic acid to organic sulfur fumes. We have also conducted “proof-of-principle” experiments described in the detailed embodiment of the invention.

For a full understanding of the present invention, references should be made to the following detailed description of the invention in its preferred embodiments, and accompanying FIGURE.

### BRIEF DESCRIPTION OF THE DRAWING

FIG. 1 is a photograph showing the retention of hair curls in both the control untreated hair sample (A) versus the fume-suppressant-treated hair sample (B).

### DESCRIPTION OF THE PREFERRED EMBODIMENTS

The preferred embodiments of the present invention will now be described.

### Example 1

### Fume Suppression: Test System

We have devised the following fume suppression assay system using the following materials:

1) A permanent wave hair care product named “Precisely Right Hard-to-Wave Base” was purchased. It is an ammonium thioglycolate (2-mercatoacetic acid) solution (hereinafter abbreviated as AT), It was stored in an amber bottle at 0-4° C. It gives off an overwhelming sulfurous odor when opened and poured off into a beaker;
2) A fume suppressant aerosol containing 12% (v/v) oleic acid in 70% ethanol;
3) An O/W (oil-in-water) emulsion system containing 4% natural cornstarch and Soybean oil (10%, v/v).
Test 1: Stripes of paper toweling (12″×8″) were placed in a tray and four (4) quarter-sized circle drawn on each with an indelible ink pen. Apply ten (10) microliters of the AT solution to the circled areas and let air dry. Repeat the application of the AT solution to the circled area to give a final of 20 microliters and let air dry. Initially there is strong sulfur odor, and even after drying there is still a significant sulfur smell for at least one hour.
Test 2: Using the same design as Test 1, apply fifty (50) microliters of AT solution to four of the circled areas. Two of the AT treated circled areas are then designated controls (C1 and C2). To the remaining two (2) AT treated circled areas (AT1 and AT2), apply a spray of the fume suppressant oleic acid-ethanol solution directly on the air dried AT treated areas.

The results of these treatments were as follows: two (2) hours after air drying, the AT (C1 and C2) control areas still gave off significant sulfur fumes. No fumes could be detected from the fume suppressant-treated AT treated areas (AT1 and AT2). This design was repeated twice with the same result.

Test 3: Apply ten (10) microliters of AT solution to a quarter-sized circled area on the right volar arm skin of a human volunteer and dab dry with paper tissue. Apply the soybean containing O/W starch gel emulsion, described above in Materials 3 to the AT treated area. The results of this test revealed that the sulfur fumes emanating from untreated skin areas were completely extinguished by the soybean oil containing O/W emulsion.
Test 4: Four (4) quarter-sized circles were drawn on absorbent paper toweling (12″×8″). Fifty microliters of AT were applied to each of the four circles and allowed to air dry. Two of the AT treated circles served as controls. To the two other AT treated circles, 75 microliters of the soybean oil O/W starch gel emulsion [3, above] was applied.

The results of this test showed that the application of the soybean oil starch gel emulsion completely abolished the sulfur odors up to several hours.

### Example 2

### Hair Wave Treatment Experiments

Hair samples from human volunteers were obtained. In order to duplicate the hair wave process on samples of hair, the AT solution was mixed with the fume suppressant 12% oleic acid containing O/W emulsion, in a one-to-one volume ratio [see Materials section above in Example 1]. The mixture was a clear colorless solution, indicating that the oleic acid was completely soluble in the AT solution at pH 8. This outcome is important as a cloudy looking solution would not be aesthetically appealing for both the Hair Salon Spa operator and customers.

Test 1: Duplicate brown hair samples were cut to a length of 3″ and placed lengthwise and dry into two separate plastic hair treatment containers. In the first container, the hair sample was covered with 5 ml of AT solution plus 5 ml of 70% ethanol. This served as a control. In the second container, the hair sample was covered with 5 ml of AT solution and another 5 ml of the fume suppressant solution (12% oleic acid in 70% ethanol) [see Materials section in Example 1 above]. There was a strong ammonia smell from both hair-treatment solutions. This is due to the ammonia fumes of the AT solution. Both plastic containers were sealed with a plastic lid, and the hair sample left in their respective solutions for thirty (30) minutes. In order to detect the reduction or elimination of the sulfur fumes, liquid samples were withdrawn from the two hair treatment containers and applied to quarter-sized circled areas drawn on absorbent paper toweling and dried. The results were detected by smelling the air-dried papers. No fumes could be detected up to 24 hours later from the fume suppressant-treated hair liquid mixture. By contrast, the control hair AT solution had a distinct sulfurous odor.
Test 2: Hair sample in Test 1 the solution in the treatment containers were drained, and the hair samples washed successively three times with 50 ml of fresh spring water, with a final rinse with 100 ml of spring water at 60° C. to remove any residual insoluble oleic acid precipitates. The water-washed hair samples were examined for evidence of sulfur odors. Only a slight odor was registered for the control AT solution soaked and washed hair. The fume suppressant treated hair sample has a slight sweet odor from the fatty acid.

### Example 3

### Hair Wave Process

An important question arose about whether the fume suppressant emulsion interferes with the hair wave process. The following experiment was designed to answer this question.

Hair samples were separated into two equal batches and designated Sample A and Sample B. The hair samples A & B were each one half inch in thickness and approximately 15 inches in length. The end of each sample was inserted into an open end of a 12×75 mm polystyrene test tube and held in place with a snap top enclosure. The hair samples were then twisted around the diameter of the tube with sufficient displacement to leave no overlaps and no gaps between the width of each hair sample. This gave about four twists per length of the tube. The ends of the hair sample were taped to the bottom of the tube.

Test: The two curled hair samples were placed in separate plastic containers. Sample A was immersed in 40 ml of permanent wave “activator” solution (ammonium thioglycolate, pH 8) containing either 70% ethanol. Sample B was immersed in 40 ml of permanent wave “activator” solution (ammonium thioglycolate, pH 8) containing 12% oleic acid in 70% ethanol. Both samples were then gently rocked for one hour at room temperature. The solutions were decanted from the plastic containers at the end of the “activator-treatment” period and replaced with 50 ml of 3% hydrogen peroxide (the neutralizer) for an additional 30 minutes at room temperature. The neutralizer was decanted and the hair samples washed successively with three 50 ml washes with spring water at 50° C. These three washes removed all traces of the oleic acid cloudiness from sample B. The hair samples were then removed from the wash containers and released from their binding to the plastic test tubes.

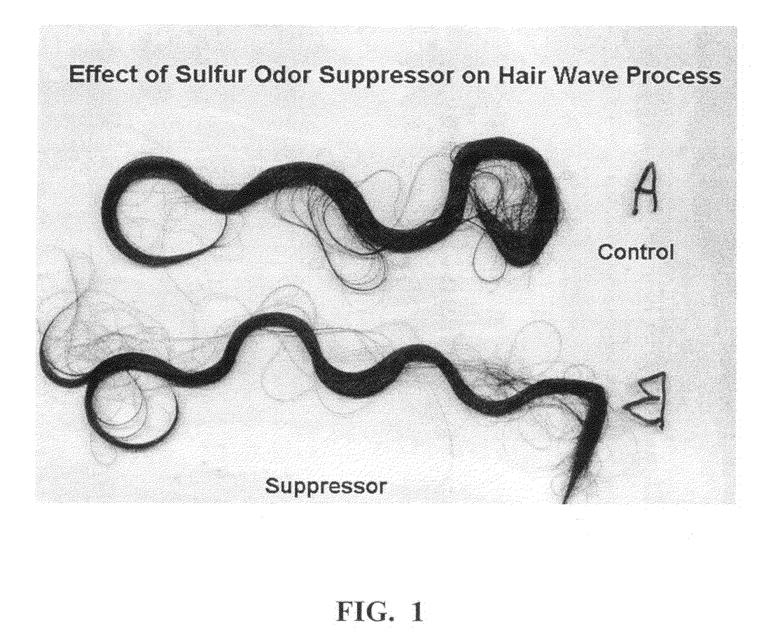

FIG. 1 shows that the hair samples retained their undulating twists following their release and even after they were stretched straight and released they retained the undulating twists to the same extent whether treated with 70% ethanol only (A) or 12% Oleic acid in 70% ethanol (B).

### Example 4

### Generality of Suppressing Organic Sulfur Fume and Other Volatile Organic Solvents by Fatty Acid Oil Treatments

This invention is not limited to only those fatty acids used in the above examples, as will be shown below in further studies aimed at understanding the chemical and physical basis of fatty acid fume suppression. Some other oils that are useful include saturated and mono-saturated fatty acids, hydrogenated vegetable oils, berry waxes and the like, and silicon oils. This relationship explains why soybean oil, consisting predominately of oleic acid and several other unsaturated oleic acid isomers with vapor pressure temperatures again well above thioglycolic acid, suppresses the sulfur fumes of thioglycolic acid.

Table 1 below lists twenty-one volatile sulfur containing organic compounds, organic acids, ethers, and nitrogen-containing compounds that have lower vapor pressure temperatures than Oleic acid. Oleic acid should suppress the sulfur fumes of each of the twenty-one compound listed below. Of course, sulfur containing organic compounds that are not liquids or oil soluble and are not expected to obey this simple relationship.

TABLE 1
Vapor Pressure temperatures of Some Organic Sulfur Compounds

| | Vapor Pressure | |
|---|---|---|
| Name of Compound | Temperature (° C.)* | |
| Ammonium Hydrogen | 33 | @ 760 mm |
| Sulfide | | |
| 2-Mercaptoethanoic | 104 | @ 100 mm |
| Acid (1) | | |
| 2-Mercaptoethanol | 35 | @ 760 mm |
| (1) | | |
| Ethanethiolic Acid | 93 | @ 760 mm |
| Thioglycol (1) | 168 | @ 760 mm |
| Dimethyl Sulfide | 36 | @ 760 mm |
| Carbon Disulfide (1) | 46.5 | @ 760 mm |
| Phosphorus | 124 | @ 760 mm |
| Thiochloride (1) | | |
| Acetic Acid (1) | 118 | @ 760 mm |
| Acetaldehyde (1) | 20 | @ 760 mm |
| Ethanol (1) | 78 | @ 760 mm |
| Ethyl Chloride (1) | 12 | @ 760 mm |
| Carbon Tetrachloride | 76.5 | @ 760 mm |
| (1) | | |
| Acetone (1) | 57 | @ 760 mm |
| Propionic Acid | 141 | @ 760 mm |
| Hydroxylamine | 110 | @ 760 mm |
| Ethyl Thiocyanic | 144 | @ 760 mm |
| Acid | | |
| Thiophene | 84 | @ 760 mm |
| Methyl Thiocyanate | 133 | @ 760 |
| Methyl | 119 | @ 760 mm |
| Isothiocyanate | | |

*Handbook of Chemistry and Physics.

The invention concerns the use of fatty acid oils, combinations of fatty acid oils that are present in a many different natural vegetable oils, hydrogenated oils, and any synthetic oils that are miscible with organic and inorganic liquids and have vapor pressure temperatures at 760 mm Hg that are greater than that of the volatile compounds found in Table 1, to suppress sulfur fumes rated from the volatile compounds found in Table 1.

There has thus been shown and described novel sulfur odor suppressant and methods for making and testing the same, of which fulfill all the objects and advantages sought therefore. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

I claim:

1. A method of suppressing low vapor pressure sulfurous fumes generated by sulfur-containing organic compounds, comprising the step of applying a liquid with a higher vapor pressure temperature than the sulfur-containing compounds to materials contaminated with the sulfur-containing organic compounds.

2. Method of claim 1, wherein the liquid is in the form of an aerosol.

3. Method of claim 1, wherein the liquid is in the form of a liquid emulsion.

4. The method of claim 1, wherein the higher vapor pressure liquid is oil selected from natural vegetable fatty acids, hydrogenated vegetable oils, and synthetic oils, which are miscible with organic and inorganic liquids, and mixtures thereof.

5. Method of suppression of low vapor pressure sulfurous fumes originating from personal hair care products, comprising the step of applying a vegetable fatty acid with a higher vapor pressure temperature than that of sulfur-containing compounds in the hair products.

6. The method of claim 5, wherein the vegetable fatty acid is 12% (v/v) oleic acid in 70% ethanol.

7. The method of claim 5, wherein the vegetable fatty acid is an O/W (oil-in-water) emulsion system containing 4% natural corn starch and soybean oil (10%, v/v).

8. Method of suppression of sulfurous fumes from permanent wave hair products using an activation solution of ammonium thioglycolate, comprising the step of applying to the activation solution a vegetable fatty acid with a higher vapor pressure temperature than that of sulfur-containing compounds in the permanent wave hair products.

9. The method of claim 8, wherein the vegetable fatty acid is 12% (v/v) oleic acid in 70% ethanol.

10. The method of claim 8, wherein the vegetable fatty acid is an O/W (oil-in-water) emulsion system containing 4% natural corn starch and soybean oil (10%, v/v).

11. The method of claim 1, wherein the liquid is selected from the group consisting of fatty acid oils, combinations of fatty acid oils that are present in a many different natural vegetable oils, hydrogenated oils, and any synthetic oils that are miscible with organic and inorganic liquids and have vapor pressure temperatures at 760 mm Hg that are greater than that of the following list of volatile compounds: ammonium hydrogen sulfide, 2-mercaptoethanoic acid, 2-mercaptoethanol, ethanethiolic acid, thioglycol, dimethyl sulfide, carbon disulfide, ethyl thiocyanic acid, thiopene, methyl thiocyanate, methyl isothiocyanate, phosphorus thiochloride, acetic acid, acetaldehyde, ethanol, ethyl chloride, carbon tetrachloride, acetone, propionic acid, hydroxylamine.
